(19) United States
(12) Patent Application Publication
(10) Pub. No.: US 2008/0269798 A1
(43) Pub. Date: Oct. 30, 2008

(54) EMBOLECTOMY DEVICE

(75) Inventors: Kamal Ramzipoor, Fremont, CA (US); Ajitkumar B. Nair, Fremont, CA (US); Riten Parikh, San Jose, CA (US); Andrew Huffmaster, Fremont, CA (US); Mehran Bashiri, San Carlos, CA (US); Pete Phong Pham, Fremont, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/171,033

(22) Filed: Jul. 10, 2008

Related U.S. Application Data
(62) Division of application No. 10/664,134, filed on Sep. 17, 2003.

(51) Int. Cl. A61M 1/34

(52) U.S. Cl. 606/200; 604/35

(57) ABSTRACT

An embodiment is a catheter comprising a first elongate shaft having a proximal end, a distal end and a first lumen therethrough, a wire having a proximal end and a distal end at least partially disposed in the first elongate shaft, the distal end extending distally from the first elongate shaft, and a motion control apparatus connected to the proximal end of the wire, further comprising a device attached to the distal end of the wire for changing the shape of an embolus, wherein the device is configured to change the shape of the embolus to unclog a distal catheter lumen.

### RELATED APPLICATIONS

This application is a divisional application of U.S. patent application Ser. No. 10/664,134 filed Sep. 17, 2003.

### FIELD OF THE INVENTION

The present invention relates generally to the field of intravascular devices. More specifically, the present invention pertains to embolectomy devices for aspirating foreign bodies within a body lumen.

### BACKGROUND OF THE INVENTION

There are a number of situations in the practice of medicine where it is desirable to remove an embolus from a patient's vasculature. If an embolus is not removed it may travel to the neural vasculature, for example, and cause severe trauma. Many prior art embolectomy devices require a retrieval portion to be placed downstream or distal the embolus. This is not always practical or desirable. Other prior art embolectomy devices may require the use of a significant vacuum to remove the embolectomy. This may cause the collapse of a portion of the vasculature and result in trauma.

### SUMMARY OF THE INVENTION

In one embodiment of an embolectomy device, a first catheter having an expandable tip may be disposed inside of a second catheter which constrains the tip. The proximal end of either the first or second catheters may be fluidly attached to a vacuum source. The tip may be expanded by moving the first catheter distally relative the second catheter. An embolus may then be urged into the tip by operating the vacuum source.

In another embodiment of an embolectomy device, a first catheter having an expandable tip may be disposed inside of a second catheter which constrains the tip. A clot pulling device may be disposed within the second catheter. The tip may be expanded by moving the first catheter distally relative the second catheter. The clot pulling device may be operated to urge an embolus into the expanded tip.

In another embodiment, a clot unclogging or fragmenting device may be disposed in a catheter, which may be fluidly connected to a vacuum source. The unclogging or fragmenting device may be connected to a motion control apparatus by a wire disposed in a lumen of the catheter. The unclogging or fragmenting device may be operated to open the tip of a catheter blocked by the clot burden or to fragment an embolus, which may then be drawn into a catheter lumen by operation of the vacuum source. The catheter may have a lumen connected to an irrigation source.

The above summary of some embodiments is not intended to describe each disclosed embodiment or every implementation of the present invention. The figures and detailed description which follow more particularly exemplify these embodiments.

### BRIEF DESCRIPTION OF THE DRAWINGS

The invention may be more completely understood in consideration of the following detailed description of various embodiments of the invention in connection with the accompanying drawings in which:

FIG. 1a depicts an embolectomy device 300 disposed in a body lumen.

FIG. 1b depicts an embolectomy device 400 disposed in a body lumen.

FIG. 1c depicts an embolectomy device 500 disposed in a body lumen.

FIG. 2a depicts a retrieval catheter 204 of embolectomy device 200.

FIG. 2b depicts a guide catheter 206 of embolectomy device 200.

FIG. 2c depicts embolectomy device 200.

FIG. 2d depicts embolectomy device 200.

FIG. 3 depicts an embolectomy device 100 disposed in a vascular lumen.

### DETAILED DESCRIPTION

The following detailed description should be read with reference to the drawings, in which like elements in different drawings are numbered identically. The drawings which are not necessarily to scale, depict selected embodiments and are not intended to limit the scope of the invention.

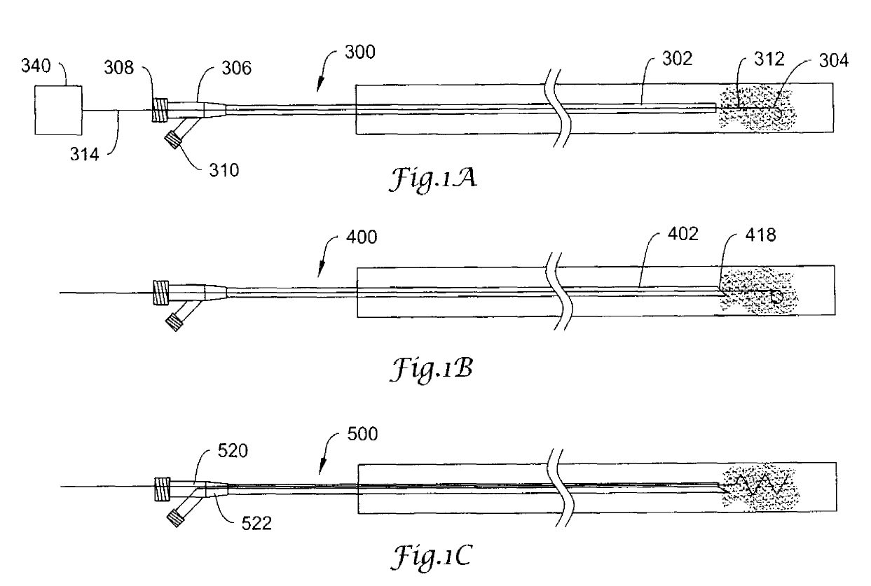

FIG. 1a depicts an embolectomy device 300 disposed in a body lumen. Device 300 includes catheter 302 and distal device 304. Distal device 304 may be used to unclog the aspiration lumen or to fragment an embolus for aspiration. Catheter 302 may have a manifold 306 attached proximally including a first port 308 and a second port 310. Distal device 304 has a proximal end 312 attached to an elongate member 314 disposed in a lumen of catheter 302. Distal device 304 may have an arcuate shape, or may be formed into a loop, coil, paddle, whisk, zigzag, helical or other shape suitable for fragmenting an embolus. The proximal end of elongate member 314 may be free or may be attached to a motion control apparatus 340 able to impart motion along the axis of elongate member 314. The motion control apparatus 340 may impart longitudinal or radial motion or vibration to the distal end of elongate member 314. Catheter 302 may also be fluidly attached to a vacuum source.

The motion control apparatus 340 may impart a motion to distal device 304 at between 1 Hz and 150 Hz. Of course, motion at higher or lower frequencies than this are envisioned. As an example, it may be advantageous to move distal device 304 at selective intervals lower than 1 Hz only when a lumen is clogged. In addition, it may be preferable to impart a motion at up to 20 kHz. The motion control apparatus 340 may have any advantageous range of motion. One example range of motion is 17 mm. This may be done by configuring the motion control apparatus 340 to move distal device 2 mm proximally and 15 mm distal from a starting position. Another example range of motion is 120 mm, with the motion control apparatus 340 configured to move distal device 304 20 mm proximally and 100 mm distally.

FIG. 1b depicts an embolectomy device 400. Device 400 is similar to device 300 and includes a catheter 402 having an angled distal end 418.

FIG. 1c depicts an embolectomy device 500. Device 500 is similar to device 300 and includes a first lumen 520 and a second lumen 522. Elongate member 314 is disposed in first lumen 520 and the vacuum source is fluidly connected to second lumen 522. In use, embolectomy device 500 may be positioned proximate an embolus and the vacuum source may be operated. Distal device 304 may be operated, either by hand or through a motion control apparatus to unclog an aspiration or other lumen or to fragment an embolus. Distal device 304 may thereby fragment the embolus and the embolus or one or more fragments thereof is drawn into second lumen 522. Distal device 304 may alter the shape of an embolus and unclog a lumen or fragment the embolus through vibrations or pulses at the distal end of elongate member 314. In an alternative use, fluid may be irrigated through first lumen 520 or through an additional lumen. Distal device 304 may alternatively or additionally be used to unclog an embolus from a lumen by removing the embolus burden and thereby creating an open channel for more effective aspiration

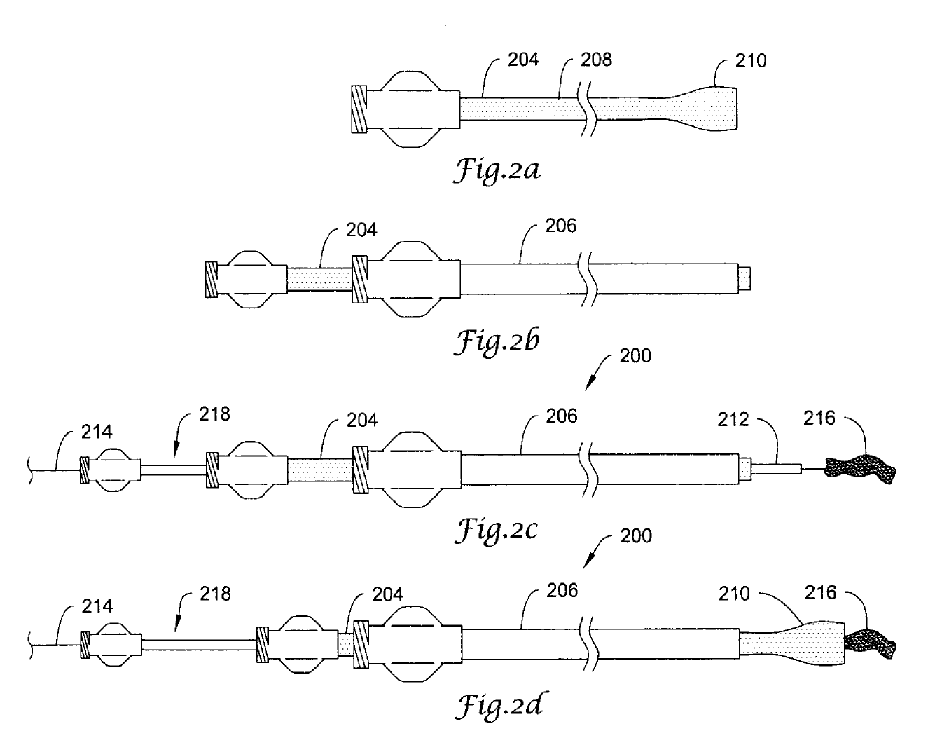

FIG. 2c depicts embolectomy device 200, which includes retrieval sheath 204 and guide catheter 206. As depicted in FIG. 2a, retrieval sheath 204 may include an expandable elongate shaft or elongate shaft 208 and expandable tip portion 210. Expandable tip portion may be formed from a shape memory polyurethane, a nitinol coiled sheet catheter, an expanding nitinol mesh or braid or other suitable material. A coiled sheet catheter may be fashioned from a flat ribbon of nitinol or other suitable material by coiling the ribbon so that proximal coils overlap and thereby constrain distal coils. When unconstrained, expandable tip portion 210 has an expanded profile and an expanded distal lumen. As shown in FIG. 2b, expandable tip portion 210 may also be constrained to fit within guide catheter 206. Embolectomy device 200 may include a clot pulling device 212, comprising an elongate member 214 and wire mesh 216 or other suitable embolus capturing device. Clot pulling device 212 may include and be disposed in a microcatheter 218. In one contemplated method, retrieval sheath 204 may be disposed in guide catheter 206 so that the distal ends are approximately even and are located proximate an embolus. Clot pulling device 212 then may be inserted through sheath 204 to capture or retain the embolus. Catheter 206 then may be moved proximally so that tip portion 210 is distally disposed of guide catheter 206 and expands as shown in FIG. 2d. Alternatively, retrieval sheath 204 may be moved distally relative guide catheter 206 to expand tip portion 210. Clot pulling device 212 may then be moved to position the embolus into the expanded tip portion 210. Retrieval sheath 204, and clot pulling device 212 may then be removed proximally from guide catheter 206. If desired, the embolic material may be removed from retrieval sheath 204 and clot pulling device 212 and these devices may be reintroduced into guide catheter 206. Of course other methods are contemplated. For instance, retrieval sheath 204 may be urged distally to cause tip portion 210 to expand and then clot pulling device 212 is inserted distally through retrieval sheath 204.

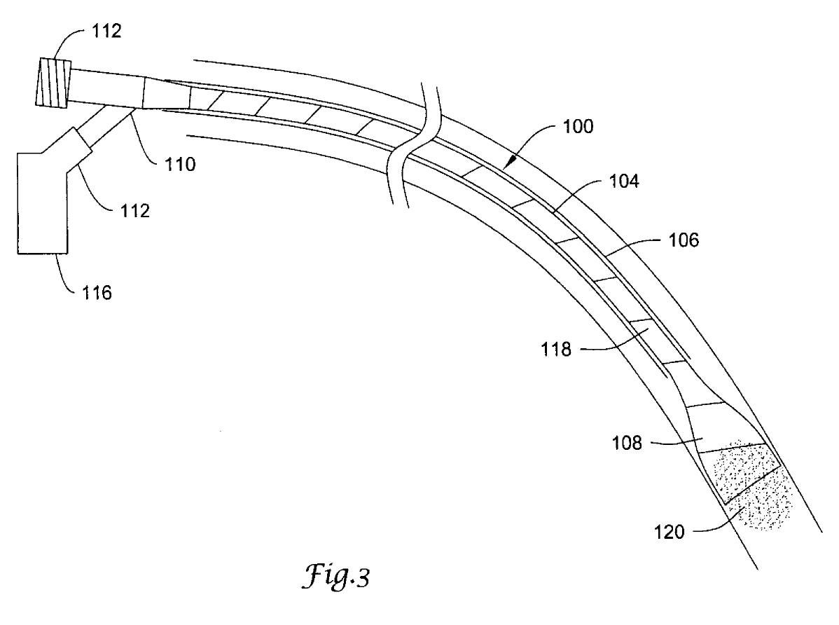

FIG. 3 depicts an embolectomy device 100 in use in a vascular lumen 102. Device 100 includes a retrieval catheter 104 and a sheath catheter 106. Retrieval catheter 104 includes lumen 118 and may have an unconstrained state where its profile has a greater cross sectional area than the profile of sheath catheter 106 or may have a tip portion 108 having an unconstrained profile having a greater cross sectional area than the profile of sheath catheter 106. Retrieval catheter 104 also has a constrained state where it may be disposed within sheath catheter 106. Retrieval catheter 104 may be fluidly coupled to a vacuum source 116 and may include a proximally positioned manifold 110 for this purpose. Manifold may include one or more axially or radially located ports 112. Retrieval catheter includes an expandable material such as a shape memory polyurethane, nitinol coiled sheet catheter, or other suitable material. In use, retrieval catheter 104 is disposed in the lumen of sheath catheter 106 and is positioned proximate an embolus 120. Retrieval catheter 104 may be extended distally or sheath catheter 106 may be retracted proximally until a desired distal portion of retrieval catheter 104, which may include tip portion 108, is in an expanded state. Vacuum source 116 may be operated to urge embolus 120 into lumen 118. Alternatively, retrieval catheter 104 having an expanded distal portion may be positioned to capture embolus 120 in lumen 118 and vacuum source 116 may be operated to secure the embolus. Once the embolus is capture, it may be removed. This may be accomplished by retracting retrieval catheter 104 proximally into sheath catheter 106 or by extending sheath catheter 106 distally. Tip portion 108 may be fully or partially disposed within sheath catheter 106. Embolectomy device 100 may then be removed from vascular lumen 102. Alternatively, retrieval catheter 104 alone may be removed distally from sheath catheter 106. In another alternative, vacuum source 116 may be operated to remove embolus 120 distally from retrieval catheter 104. In another alternative an irrigation catheter may be used to provide fluid.

Numerous advantages of the invention covered by this document have been set forth in the foregoing description. It will be understood, however, that this disclosure is, in many respects, only illustrative. Changes may be made in details, particularly in matters of shape, size, and arrangement of parts or order of steps without exceeding the scope of the invention. The invention's scope is, of course, defined in the language in which the appended claims are expressed.

1. A method comprising the steps of:
providing a catheter having a wire for fragmenting an embolus at least partially disposed within a lumen of the catheter;
positioning a distal end of the catheter proximate an embolus; and
manipulating the wire to change the shape of the embolus,
wherein the catheter has a proximal end, a distal end and a lumen extending therebetween, the lumen having a longitudinal axis extending from the proximal end to the distal end, wherein the wire has a proximal end and a distal end and a center line extending therebetween that follows the path of the wire, the wire further having a proximal region, an intermediate region and a distal region, and the wire being at least partially disposed in the lumen, wherein the intermediate region has a generally uniform width along its length, and wherein the distal region of the wire includes a distal tip having a uniform profile along a length and a region proximate the distal tip having a maximum width perpendicular to the longitudinal axis of the lumen that is greater than the width of the intermediate region of the wire and wherein the distal tip has a width that is less than that of the proximate region and wherein the intermediate region width is also less than that of the proximate region.

2. The method of claim 1 wherein the step of manipulating the wire includes the step of unclogging a lumen to assist aspiration of the embolus.

3. The method of claim 1 wherein the step of manipulating the wire includes the step of fragmenting an embolus.

4. The method of claim 1 wherein the step of manipulating the wire includes the step of direct operator manipulation of a proximal end of the wire.

5. The method of claim 4, further comprising the step of providing a handle attached to a proximal portion of the wire for direct operator manipulation.

6. The method of claim 1, wherein the embolus is located in a patient's vasculature.

7. The method of claim 1 wherein the step of positioning the distal end of the catheter includes the step of positioning the distal end of the wire within the embolus.

8. The method of claim 1 further comprising the steps of
providing a vacuum source fluidly connected to the distal end of the catheter, and operating the vacuum source.

9. The method of claim 8 further comprising the step of flushing a region proximate the embolus with a fluid.

10. The method of claim 1 wherein the distal region of the wire has a maximum width that is less than half the diameter of the blood vessel.

11. The method of claim 10 wherein the proximate region of the distal region has a curved profile parallel to the longitudinal axis.

12. The method of claim 11 wherein the maximum width of the proximate region is at least twice the width of the distal tip.

13. The method of claim 12 wherein the cross-sectional shape of the wire along the center line is a simple closed shape.

14. (canceled)

15. The method of claim 14 wherein the wire is configured to move rapidly along the longitudinal axis of the lumen proximally and distally.

16. The method of claim 13 wherein the proximate region has a first section having a first end that has a maximum thickness of material perpendicular to the longitudinal axis and a second end that abuts the distal tip, the proximate region further having a second section having a first end that abuts the first end of the first section and a second end that has a width equal to that of the intermediate region of the wire,
wherein the first section is longer than the second section.

17. The method of claim 16 wherein the first section transitions gradually from the first end of the first section to the second end of the first section.

18. The method of claim 16 wherein the first section is at least twice as long as the second section.

19. The method of claim 16 wherein the magnitude of the tangent of any angle between a first line tangent to a point on the center line in the first section and a second line, coplanar to the first line, that is tangent to a second point on the surface of the wire is less than 0.84, where the second point is defined by the intersection of the surface of the wire and a line that extends through the point on the center line and is perpendicular to the first line.

20. The method of claim 19 wherein the magnitude of the tangent is less than 0.71.

21. The method of claim 19 wherein the magnitude of the tangent is less than 0.58.

22. The method of claim 19 wherein the magnitude of the tangent is less than 0.47.

23. The method of claim 19 wherein the magnitude of the tangent is less than 0.37.

24. The method of claim 1 wherein the distal tip and the intermediate regions have approximately the same cross-sectional areas.

25. The method of claim 24 wherein the distal tip cross-sectional area is equal to that of the intermediate region.

26. A method comprising the steps of:
providing a catheter having a wire for fragmenting an embolus at least partially disposed within a lumen having a diameter and an elongate axis, the wire having an average thickness;
positioning a distal end of the catheter proximate an embolus;
manipulating the wire by hand to change the shape of the embolus;
providing a vacuum source fluidly connected to the distal end of the catheter; and
operating the vacuum source,
wherein the distal section of the wire has a width perpendicular to the elongate axis that is greater than the average thickness of the wire and less than the diameter of the lumen.

27. The method of claim 26 wherein the cross-sectional shape of the wire is a simple closed shape.

28. The method of claim 26 wherein the width of the distal section of the wire is less than half the diameter of the lumen.

29. The method of claim 26 wherein the wire has a distal tip where the thickness is equal to or less than the average thickness along and wherein the thickness of the distal tip is uniform along a length of the wire.

30. The method of claim 1 wherein the wire is free from attachment to a motion control apparatus.

31. The method of claim 15 wherein the wire is configured to move at no more than 20 Hz.
